SEQUENCE LISTING (6 sequences)

SEQ ID NO: 1 - DNA, 921 bp, Homo sapiens
  source 1..921: mol_type = unassigned DNA
atggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatga

SEQ ID NO: 2 - protein, 306 aa, Homo sapiens
MASPGCLWLLAVALLPWTCASRALQHLDPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 3 - DNA, 546 bp, synthetic construct
  source 1..546: mol_type = other DNA
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 4 - DNA, 270 bp, synthetic construct
  source 1..270: mol_type = other DNA
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 5 - DNA, 5612 bp, synthetic construct
  source 1..5612: mol_type = other DNA
gcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcaattcacgcgccggtaccgaattcacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactaccggtatggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 6 - DNA, 5706 bp, synthetic construct
  source 1..5706: mol_type = other DNA
ctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatggcgtcgcccggctgcctgtggctcttggctgtggctctcctgccatggacctgcgcttctcgggcgctgcagcatctggacccgccggcgccgctgccgttggtgatctggcatgggatgggagacagctgttgcaatcccttaagcatgggtgctattaaaaaaatggtggagaagaaaatacctggaatttacgtcttatctttagagattgggaagaccctgatggaggacgtggagaacagcttcttcttgaatgtcaattcccaagtaacaacagtgtgtcaggcacttgctaaggatcctaaattgcagcaaggctacaatgctatgggattctcccagggaggccaatttctgagggcagtggctcagagatgcccttcacctcccatgatcaatctgatctcggttgggggacaacatcaaggtgtttttggactccctcgatgcccaggagagagctctcacatctgtgacttcatccgaaaaacactgaatgctggggcgtactccaaagttgttcaggaacgcctcgtgcaagccgaatactggcatgaccccataaaggaggatgtgtatcgcaaccacagcatcttcttggcagatataaatcaggagcggggtatcaatgagtcctacaagaaaaacctgatggccctgaagaagtttgtgatggtgaaattcctcaatgattccattgtggaccctgtagattcggagtggtttggattttacagaagtggccaagccaaggaaaccattcccttacaggagacctccctgtacacacaggaccgcctggggctaaaggaaatggacaatgcaggacagctagtgtttctggctacagaaggggaccatcttcagttgtctgaagaatggttttatgcccacatcataccattccttggatgacctgcaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattcgcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgct

(19) United States
(12) Patent Application Publication
(10) Pub. No.: US 2025/0382593 A1
(43) Pub. Date: Dec. 18, 2025

(54) Adeno-Associated Virus Delivery of CLN1 Polynucleotide

(71) Applicant: RESEARCH INSTITUTE AT NATIONWIDE CHILDREN'S HOSPITAL, Columbus, OH (US)

(72) Inventors: Kathrin Christine Meyer, Columbus, OH (US); Shibi Likhite, Columbus, OH (US)

(21) Appl. No.: 18/880,004

(22) PCT Filed: Jul. 5, 2023
(86) PCT No.: PCT/US23/69625

§ 371 Date: Dec. 30, 2024

Related U.S. Application Data
(60) Provisional application No. 63/367,752, filed on Jul. 6, 2022.

(51) Int. Cl. C12N 9/16; A61K 38/46; A61K 48/00; A61P 25/00; C12N 15/86

(52) U.S. Cl. CPC C12N 9/16; A61K 38/465; A61K 48/005; A61P 25/00; C12N 15/86; C12Y 301/02022; C12N 2750/14143

(57) ABSTRACT

The present disclosure relates to recombinant adeno-associated virus (rAAV) delivery of a neuronal ceroid lipofuscinosis neuronal 1 (CLN1) polynucleotide. The disclosure provides rAAV and methods of using the rAAV for CLN1 gene therapy of the neuronal ceroid lipofuscinosis CLN1-Batten Disease.

This application claims priority to U.S. Provisional Patent Application No. 63/367,752, filed Jul. 6, 2022, which is incorporated by reference in this entirety.

### INCORPORATION BY REFERENCE OF THE SEQUENCE LISTING

This application contains, as a separate part of the disclosure, a Sequence Listing in computer-readable form which is incorporated by reference in its entirety and identified as follows: 56756_Seqlisting.XML; Size: 58.419 bytes; Created: Jul. 3, 2023.

### FIELD

The present disclosure relates to recombinant adeno-associated virus (rAAV) delivery of a neuronal ceroid lipofuscinosis neuronal 1 (CLN1) polynucleotide. The disclosure provides rAAV and methods of using the rAAV for CLN1 gene therapy of the neuronal ceroid lipofuscinosis (NCL) or CLN1-Batten Disease.

### BACKGROUND

Neuronal ceroid lipofuscinoses (NCLs) are a group of severe neurodegenerative disorders, which are collectively referred to as Batten disease. These disorders affect the nervous system and typically cause worsening problems with vision, movement, and thinking ability. The different NCLs are distinguished by their genetic cause.

CLN1-Batten disease is an inherited autosomal recessive disorder caused by mutations in the CLN1 gene (also known as the PPT1 gene). The CLN1 gene encodes a 306 amino acid protein, palmitoyl protein thioesterase 1 (PPT1). PPT1 is a lysosomal enzyme involved in the removal of palmitate residues from proteins. PPT1 is also associated with important cellular pathways, including synaptogenesis and synaptic maintenance, endosomal trafficking and lipid metabolism (Johnson et al., Nat Rev Neurol. 2019 March; 15 (3): 161-178).

Classic onset of CLN1-Batten disease is in the first year of life, with irritability, developmental arrest and rapid regression, deceleration of head circumference growth, hypotonia. myoclonic seizures and progressive vision loss with optic nerve atrophy (Johnson et al., Nat Rev Neurol. 2019 March; 15 (3): 161-178). However, some children with CLN1 mutations develop Batten disease symptoms after infancy, around age 5 or 6. Children with CLN1 disease have decreased muscle tone (hypotonia), intellectual and motor disability, and rarely are able to speak or walk. Some affected children develop repetitive hand movements. By age 2, individuals with this condition often have muscle twitches (myoclonus), recurrent seizures (epilepsy), and vision loss.

Currently, there are no therapies that can reverse the symptoms of CLN1-Batten Disease. Seizures can sometimes be reduced or controlled with antiseizure drugs. Thus, there is a need in the art for treatments for CLN1-Batten Disease.

### SUMMARY

The present disclosure provides methods and products for treating CLN1-Batten Disease. Provided herein are methods and products for CLN1 gene therapy using recombinant AAV. The methods involve delivery of a CLN1 polynucleotide to a subject using rAAV as a gene delivery vector.

Provided herein are polynucleotides comprising a nucleic acid sequence encoding the CLN1 polypeptide. In some embodiments, the CLN1 polypeptide comprises an amino acid sequence at least 90% identical to SEQ ID NO: 2. In some embodiments, the CLN1 polypeptide comprises the amino acid sequence of SEQ ID NO: 2. In some embodiments, the polynucleotide sequence encoding the CLN1 polypeptide comprises a nucleotide sequence that is at least 90% identical to the nucleotide sequence of SEQ ID NO: 1. In some embodiments, the polynucleotide sequence encoding the CLN1 polypeptide comprises the nucleotide sequence of SEQ ID NO: 1.

In various embodiments, the polynucleotides disclosed herein comprise a nucleotide sequence at least 90% identical to nucleotides 980-3062 of SEQ ID NO: 5. In some embodiments, the polynucleotide comprises nucleotides 980-3062 of SEQ ID NO: 5. In various embodiments, the polynucleotides disclosed herein comprise a nucleotide sequence at least 90% identical to nucleotides 610-2786 of SEQ ID NO: 6. In some embodiments, the polynucleotide comprises nucleotides 610-2786 of SEQ ID NO: 6.

In various embodiments, the polynucleotide further comprises the P456 promoter or the chicken β-actin (CB) promoter. In some embodiments, the polypeptide comprises s P546 promoter comprising the sequence of SEQ ID NO: 3 and a nucleic acid sequence encoding the CLN1 polypeptide of SEQ ID NO: 2. In some embodiments, the polynucleotide comprises a CB promoter comprising the sequence of SEQ ID NO: 4 and a nucleic acid sequence encoding the CLN1 polypeptide of SEQ ID NO: 2.

Also provided herein are recombinant adeno-associated virus (rAAV) vectors comprising any of the polynucleotides disclosed herein. In some embodiments, the rAAV is of the serotype AAV1, AAV2, AAV3, AAV4, AAV5, AAV6, AAV7, AAV8, AAV9, AAVRH10, AAVrh74, AAV11, AAV12, AAV13 or Anc80, AAV7m8 and their derivatives. In addition, viral particles comprising any of the disclosed polynucleotides or rAAV vectors are provided. The rAAV with self-complementary or single-stranded genomes are also provided.

Also provided are recombinant adeno-associated virus (rAAV) viral particles encoding a CLN1 polypeptide, comprising an rAAV9 genome comprising in 5′ to 3′ order: a P546 promoter, and a polynucleotide encoding the CLN1 polypeptide.

Further provided are recombinant adeno-associated virus (rAAV) viral particle encoding a CLN1 polypeptide, comprising an rAAV9 genome comprising in 5′ to 3′ order: a CB promoter, and a polynucleotide encoding the CLN1 polypeptide.

Further provided are self-complementary recombinant adeno-associated virus (scAAV) comprising the any of the polynucleotides disclosed herein, any of the rAAV disclosed herein, or any of the rAAV particles disclosed herein. In some embodiments, the scAAV comprises a single stranded genome.

Also provided are compositions comprising the any of the nucleotides described herein, any of the rAAV viral particles described herein, or any of the scAAV described herein and a pharmaceutically acceptable excipient, carrier, or diluent. In some embodiments the excipient comprises a non-ionic low osmolar compound.

Still further provided are methods of treating CLN1-Batten Disease in an individual comprising administering to the individual a composition comprising any of the polynucleotides described herein, any of the rAAV vectors described herein, any of the viral particles described herein, any of the scAAVs described herein, or any of the compositions described herein.

Also provided are compositions for treating CLN1-Batten Disease in a subject, comprising a therapeutically effective amount of any of the polynucleotides described herein, any of the rAAV vectors described herein, any of the viral particles described herein, any of the scAAVs described herein, or any of the composition described herein for treating CLN1-Batten Disease.

The disclosure also provides use of a therapeutically effective amount of any of the polynucleotides described herein, any of the rAAV vectors described herein, any of the viral particles described herein, any of the scAAVs described herein, any of the scAAVs described herein, or any of the compositions described herein for the preparation of a medicament for treating CLN1-Batten Disease.

In any of the methods, uses or compositions for treating CLN1-Batten Disease provided, any of the compositions, medicaments, rAAV vectors, viral particles scAAV, and/or polynucleotides are formulated for administration via an intrathecal route, an intracerebroventricular route, an intraperenchymal route, an intravenous route, or a combination thereof.

In any of the methods, uses or compositions for treating CLN1-Batten Disease provided, about 1×10¹³ to about 1×10¹⁵ vg of the scAAV or rAAV viral particle is administered.

In any of the methods, uses or compositions for treating CLN1-Batten Disease provided, further comprising placing the individual in the Trendelenberg position after administering of the scAAV, rAAV viral particle, polynucleotide or the composition.

The headings herein are for the convenience of the reader and not intended to be limiting.

The use of ‘may’ and ‘can’ herein is to describe the various embodiments that are included within the claims, and not to indicate uncertainty about the scope of the claims.

### BRIEF DESCRIPTION OF THE DRAWINGS

FIG. 1 provides a schematic of an exemplary gene cassette, pscAAV9.P546.CLN1.Kan (SEQ ID NO: 5).

FIG. 2 provides an annotated nucleotide sequence of pAAV9.P546.CLN1.Kan (SEQ ID NO: 5).

FIG. 3 provides a schematic of an exemplary gene cassette, pscAAV9.CB.CLN1.Kan (SEQ ID NO: 6).

FIG. 4 provides an annotated nucleotide sequence of pAAV9.CB.CLN1.Kan (SEQ ID NO: 6).

### DETAILED DESCRIPTION

The present disclosure provides methods and products for treating CLN1-Batten Disease. The methods involve delivery of a CLN1 polynucleotide to a subject using rAAV as a gene delivery vector.

In one aspect, the invention provides methods for the intrathecal administration (i.e., administration into the space under the arachnoid membrane of the brain or spinal cord) of a polynucleotide encoding CLN1 to a patient comprising administering a rAAV9 with a genome including the polynucleotide. In some embodiments, the rAAV9 genome is a self-complementary genome. In other embodiments, the rAAV9 genome is a single-stranded genome.

The methods deliver the polynucleotide encoding CLN1 to the brain and spinal cord of the patient (i.e., the central nervous system of the patient). Some target areas of the brain contemplated for delivery include, but are not limited to, the motor cortex and the brain stem. Some target cells of the central nervous system contemplated for delivery include, but are not limited to, nerve cells and glial cells. Examples of glial cells are microglial cells, oligodendrocytes and astrocytes.

### AAV Gene Therapy

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including 145 nucleotide inverted terminal repeat (ITRs) and may be used to refer to the virus itself or derivatives thereof. The term covers all subtypes and both naturally occurring and recombinant forms, except where specified otherwise. There are multiple serotypes of AAV. The serotypes of AAV are each associated with a specific clade, the members of which share serologic and functional similarities. Thus, AA Vs may also be referred to by the clade. For example, AAV9 sequences are referred to as “clade F” sequences (Gao et al., J. Virol., 78:6381-6388 (2004). The present disclosure contemplates the use of any sequence within a specific clade, e.g., clade F. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); the AAV-11 genome is provided in Virology, 330 (2): 375-383 (2004); portions of the AAV-12 genome are provided in Genbank Accession No. DQ813647; portions of the AAV-13 genome are provided in Genbank Accession No. EU285562. The sequence of the AAV rh.74 genome is provided in see U.S. Pat. No. 9,434,928, incorporated herein by reference. The sequence of the AAV-B1 genome is provided in Choudhury et al., Mol. Ther., 24 (7): 1247-1257 (2016). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. In some instances, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

The term “AAV” as used herein refers to the wild type AAV virus or viral particles. The terms “AAV.” “AAV virus,” and “AAV viral particle” are used interchangeably herein. The term “rAAV” refers to a recombinant AAV virus or recombinant infectious, encapsulated viral particle. The terms “rAAV.” “rAAV virus,” and “rAAV viral particle” are used interchangeably herein.

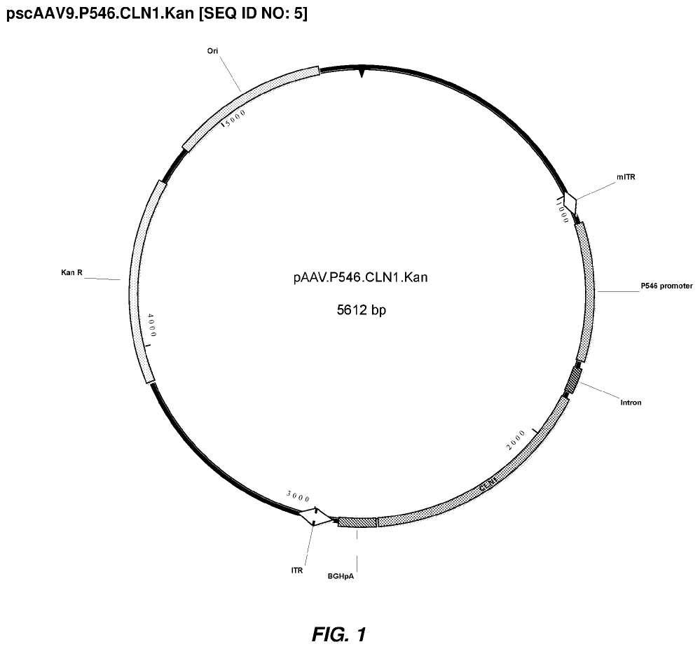

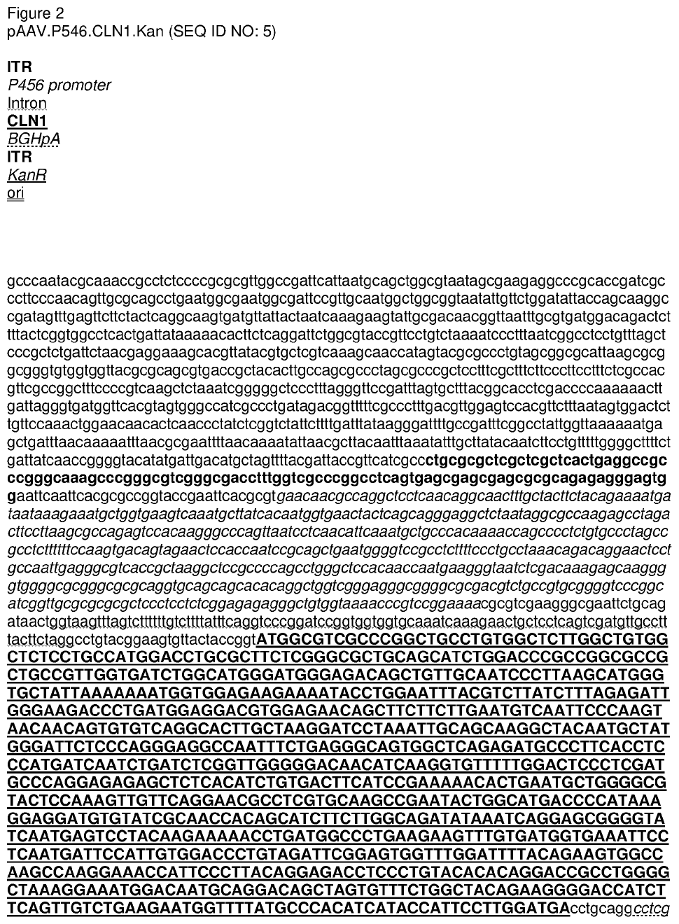

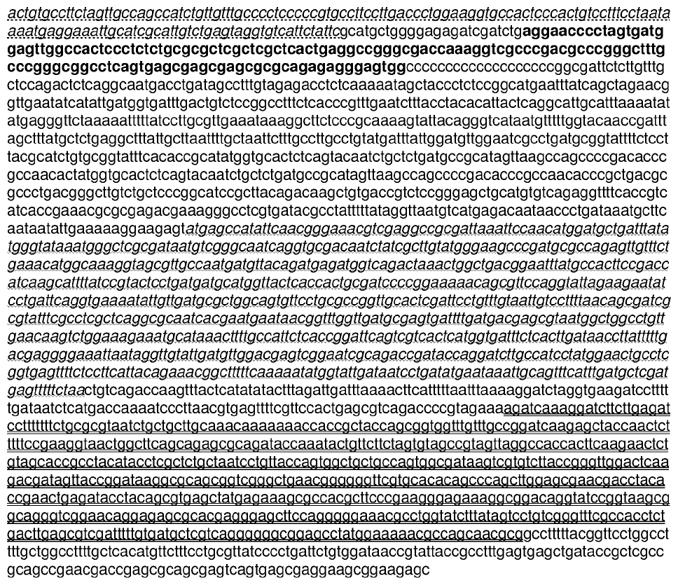

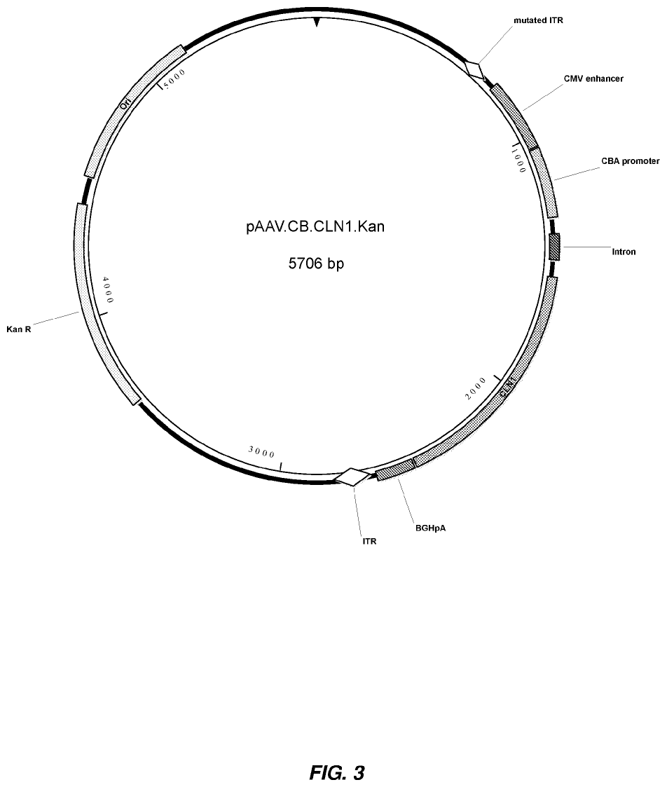

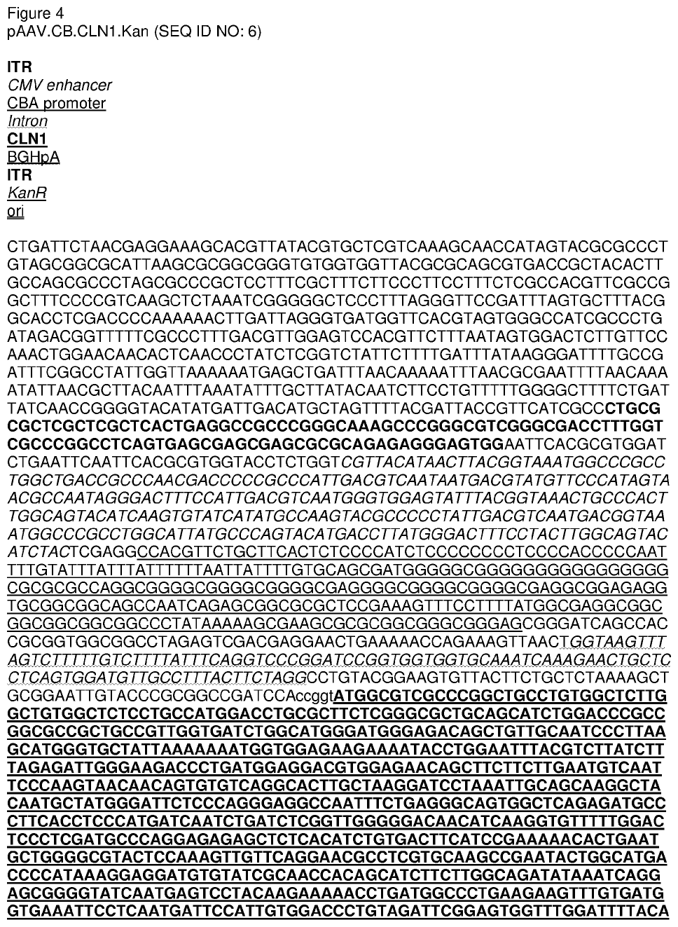

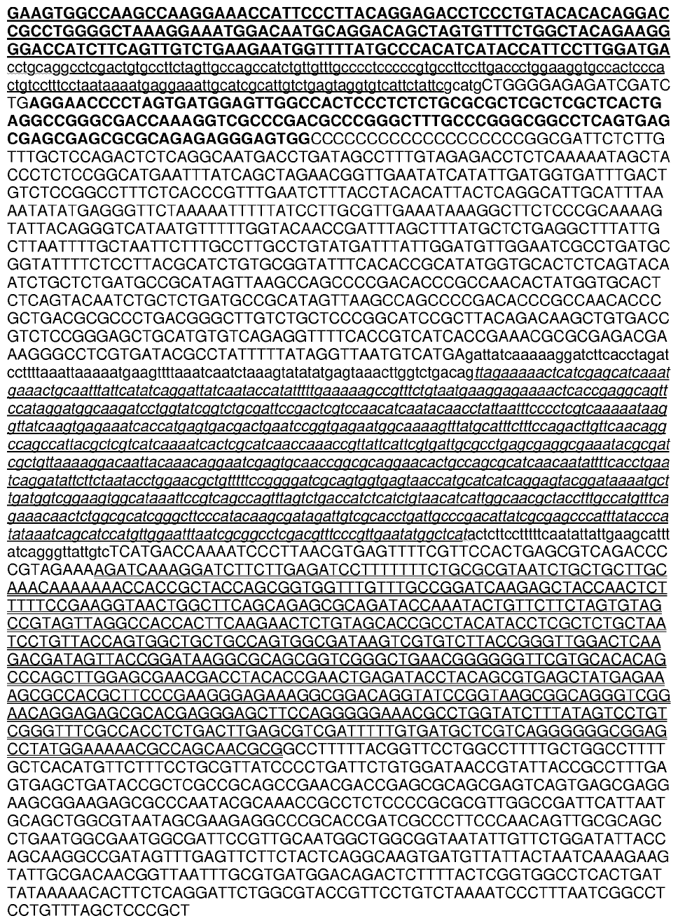

The term “rAAV genome” refers to a polynucleotide sequence that is derived from a native AAV genome that has been modified. In some embodiments, the rAAV genome has been modified to remove the native cap and rep genes. In some embodiment, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest (e.g., a CLN1-encoding polynucleotide) flanked on the 5′ and 3′ ends by inverted terminal repeat (ITR). In some embodiments, the rAAV genome comprises a “gene cassette.” Exemplary gene cassettes are set out in FIGS. 1 and 3 and the nucleic acid sequence of SEQ ID NO: 5 and 6 respectfully. The rAAV genome can be a self-complementary (sc) genome, which is referred to herein as “scAAV genome. Alternatively, the rAAV genome can be a single-stranded (ss) genome, which is referred to herein as “ssAAV genome.”

The term “scAAV” refers to a rAAV virus or rAAV viral particle comprising a self-complementary genome. The term “ssAAV” refers to an rAAV virus or rAAV viral particle comprising a single-stranded genome.

rAAV genomes provided herein comprise a polynucleotide encoding a CLN1 polypeptide. CLN1 polypeptides comprise the amino acid sequence set out in SEQ ID NO: 2 or a polypeptide with an amino acid sequence that is at least: 90%, 91%, 92%, 93%, 94%, 95%, 96%, 97%, 98%, or 99% identical to the amino acid sequence set forth in SEQ ID NO: 2, and which encodes a polypeptide with CLN1 activity (e.g., at least one of: increasing clearance of lysosomal auto fluorescent storage material, reducing or slowing lysosomal accumulation of ATP synthase subunit C, and reducing activation of astrocytes and microglia in a patient when treated as compared to e.g., the patient prior to treatment).

rAAV genomes provided herein, in some cases, comprise a polynucleotide encoding a CLN1 polypeptide wherein the polynucleotide has the nucleotide sequence set out in SEQ ID NO: 1, or a polynucleotide at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 1 and encodes a polypeptide with CLN1 activity (e.g., at least one of increasing clearance of lysosomal auto fluorescent storage material, reducing or slowing lysosomal accumulation of ATP synthase subunit C, and reducing activation of astrocytes and microglia in a patient when treated as compared to e.g., the patient prior to treatment).

rAAV genomes provided herein, in some embodiments, comprise a polynucleotide sequence that encodes a polypeptide with CLN1 activity and that hybridizes under stringent conditions to the nucleic acid sequence of SEQ ID NO: 1, or the complement thereof. The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989).

The rAAV genomes provided herein, in some embodiments, comprise one or more AAV ITRs flanking the polynucleotide encoding a CLN1 polypeptide. The CLN1 polynucleotide is operatively linked to transcriptional control elements (including, but not limited to, promoters, enhancers and/or polyadenylation signal sequences) that are functional in target cells to form a gene cassette. Examples of promoters are the P546 promoter and the chicken-β-actin promoter. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-la promoter, the hemoglobin promoter, and the creatine kinase promoter.

Provided herein are P546 promoter sequences, for example the P546 promoter sequence set out in SEQ ID NO: 3, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence set forth in SEQ ID NO: 3 that are promoters with P546 transcription promoting activity.

Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Examples of inducible promoters include, but are not limited to a metallothionine promoter, a glucocorticoid promoter, a progesterone promoter, and a tetracycline-regulated promoter. The gene cassette may also include intron sequences to facilitate processing of a CLN1 RNA transcript when expressed in mammalian cells. One example of such an intron is the SV40 intron.

Conservative nucleotide substitutions in the rAAV9 genome including, but not limited to, in the gene cassette in the rAAV9 genome, are contemplated. For example, a CLN1 cDNA in a gene cassette may have 80%, 81%, 82%, 83%, 84%, 85%, 86%, 87%, 88%, 89%, 90%, 91%, 92%, 93%, 94%, 95%, 96%, 97%, 98% or 99% sequence identity to the CLN1 nucleotide sequence, such as the nucleotide sequence of SEQ ID NO: 1 that encodes a protein that retains CLN1 activity.

The terms “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. The percentage identity of the sequences can be determined by techniques known in the art. For example, homology can be determined by a direct comparison of the sequence information between two polypeptide molecules by aligning the sequence information and using readily available computer programs such as ALIGN, ClustalW2 and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, Gen-Bank+EMBL+DDBJ+PDB+GenBank CDS translations+Swiss protein+Spupdate+PIR.

“Packaging” refers to a series of intracellular events that result in the assembly and encapsidation of an AAV particle. The term “production” refers to the process of producing the rAAV (the infectious, encapsulated rAAV particles) by the packing cells.

AAV “rep” and “cap” genes refer to polynucleotide sequences encoding replication and encapsidation proteins, respectively, of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allows AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

The rAAV genomes provided herein lack AAV rep and cap DNA. AAV DNA in the rAAV genomes (e.g., ITRs) contemplated herein may be from any AAV serotype suitable for deriving a recombinant virus including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV rh. 10, AAV rh.74 and AAV-B1. As noted above, the nucleotide sequences of the genomes of various AAV serotypes are known in the art. rAAV with capsid mutations, are also contemplated. See, for example, Marsic et al., Molecular Therapy, 22 (11): 1900-1909 (2014). Modified capsids herein are also contemplated and include capsids having various post-translational modifications such as glycosylation and deamidation. Deamidation of asparagine or glutamine side chains resulting in conversion of asparagine residues to aspartic acid or isoaspartic acid residues, and conversion of glutamine to glutamic acid or isoglutamic acid is contemplated in rAAV capsids provided herein. See, for example, Giles et al., Molecular Therapy, 26 (12): 2848-2862 (2018). Modified capsids herein are also contemplated to comprise targeting sequences directing the rAAV to the affected tissues and organs requiring treatment.

DNA plasmids provided herein comprise rAAV genomes described herein. The DNA plasmids are transferred to cells permissible for infection with a helper virus of AAV (e.g., adenovirus, El-deleted adenovirus or herpesvirus) for assembly of the rAAV genome into infectious viral particles with AAV9 capsid proteins. Techniques to produce rAAV, in which an rAAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs. Production of pseudotyped rAAV is disclosed in, for example, WO 01/83692 which is incorporated by reference herein in its entirety. In various embodiments, AAV capsid proteins may be modified to enhance delivery of the recombinant rAAV. Modifications to capsid proteins are generally known in the art. See, for example, US 2005/0053922 and US 2009/0202490, the disclosures of which are incorporated by reference herein in their entirety.

The rAAV genomes provided herein, in some embodiments, comprise one or more AAV ITRs flanking the transgene polynucleotide sequence. The transgene polynucleotide sequence is operatively linked to transcriptional control elements (including, but not limited to, promoters, enhancers and/or polyadenylation signal sequences) that are functional in target cells to form a gene cassette. Examples of promoters are the pIRF promoter, the P546 promoter comprising the polynucleotide sequence set forth in SEQ ID NO: 3, and the chicken-β-actin promoter (CB or CBA) comprising the polynucleotide sequence set forth in SEQ ID NO: 4. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-la promoter, the hemoglobin promoter, and the creatine kinase promoter.

A method of generating a packaging cell is to create a cell line that stably expresses all the necessary components for rAAV production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

Further provided herein are packaging cells that produce infectious rAAV. In one embodiment packaging cells may be stably transformed cancer cells such as HeLa cells, 293 cells, and PerC.6 cells (a cognate 293 line). In another embodiment, packaging cells are cells that are not transformed cancer cells such as low passage 293 cells (human fetal kidney cells transformed with E1 of adenovirus), MRC-5 cells (human fetal fibroblasts), WI-38 cells (human fetal fibroblasts), Vero cells (monkey kidney cells), and FRhL-2 cells (rhesus fetal lung cells).

Also provided herein are rAAV (e.g., infectious encapsidated rAAV particles) comprising a rAAV genome of the disclosure. The genomes of the rAAV lack AAV rep and cap DNA, that is, there is no AAV rep or cap DNA between the ITRs of the genomes of the rAAV. The rAAV genome can be a self-complementary (sc) genome. A rAAV with a sc genome is referred to herein as a scAAV. The rAAV genome can be a single-stranded (ss) genome. An rAAV with a single-stranded genome is referred to herein as an ssAAV.

An exemplary rAAV provided herein is the scAAV named “scAAV9.P546.CLN1.” The scAAV9.P546.CLN1 contains an scAAV9 genome comprising a human CLN1 cDNA under the control of a truncated Methyl CpG binding protein 2 (MeCP2) promoter herein referred to as the P546 promoter (SEQ ID NO: 3). The scAAV also comprises a SV40 Intron (upstream of human CLN1 cDNA) and Bovine Growth Hormone polyadenylation (BGH Poly A) terminator sequence (downstream of human CLN1 cDNA). The sequence of this scAAV9.P546.CLN1 gene cassette is set out in SEQ ID NO: 5. The scAAV9 genome is packaged in AAV9 capsid and includes AAV2 ITRs (one ITR upstream of the P546 promoter and the other ITR downstream of the BGH Poly A terminator sequence).

Another exemplary rAAV provided herein is the scAAV named “scAAV9.CB.CLN1.” The scAAV9.CB.CLN1 contains an scAAV9 genome comprising a human CLN1 cDNA under the control of a CMV-enhancer chicken β-actin (CB) promoter herein referred (SEQ ID NO: 4). The scAAV also comprises a SV40 Intron (upstream of human CLN1 cDNA) and Bovine Growth Hormone polyadenylation (BGH Poly A) terminator sequence (downstream of human CLN1 cDNA). The sequence of this scAAV9.CB.CLN1 gene cassette is set out in SEQ ID NO: 6. The scAAV9 genome is packaged in AAV9 capsid and includes AAV2 ITRs (one ITR upstream of the P546 promoter and the other ITR downstream of the BGH Poly A terminator sequence).

The rAAV may be purified by methods standard in the art such as by column chromatography or cesium chloride gradients. Methods for purifying rAAV from helper virus are known in the art and include methods disclosed in, for example, Clark et al., Hum. Gene Ther., 10 (6): 1031-1039 (1999); Schenpp and Clark, Methods Mol. Med., 69:427-443 (2002); U.S. Pat. No. 6,566,118 and WO 98/09657.

Compositions comprising rAAV are also provided. Compositions comprise a rAAV encoding a CLN1 polypeptide. Compositions may include two or more rAAV encoding different polypeptides of interest. In some embodiments, the rAAV is scAAV or ssAAV.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgl/mL, an osmolality by vapor-pressure osmometry of about 322mOsm/kg water, an osmolarity of about 273mOsm/L, an absolute viscosity of about 2.3cp at 20° C. and about 1.5cp at 37° C., and a specific gravity of about 1.164 at 37° C. Exemplary compositions comprise about 20 to 40% non-ionic, low-osmolar compound or about 25% to about 35% non-ionic, low-osmolar compound. An exemplary composition comprises scAAV formulated in 20 mM Tris (pH8.0), 1 mM MgCl2, 200 mM NaCl. 0.001% poloxamer 188 and about 25% to about 35% non-ionic, low-osmolar compound. Another exemplary composition comprises scAAV formulated in 1XPBSand 0.001% Pluronic F68.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include from about 1×10¹¹, about 1×10¹², about 1×10¹³, about 5×10¹³, about 1.2×10¹⁴, about 1.5×10¹⁴, about 2×10¹⁴, about 1×10¹⁵, to about 1×10¹⁶, or more total viral genomes. Dosages of about 1×10¹¹ to about 1×10¹⁵ vg. 1×10¹³ to about 1×10¹⁵ vg. about 1×10¹³ to about 5×10¹³, about 1×10¹³ to about 2×10¹⁴ vg. about 5×10¹³ to about 1×10¹⁴ vg, and about 1.5×10¹⁴ to about 2×10¹⁴ vg are also contemplated. In various embodiments, the dose of a composition comprising a rAAV provided herein is based on the size and growth rate of the nervous system and the cerebrospinal fluid. The dose is not based on the body weight but on absolute dose per patient.

Exemplary compositions comprise an agent to increase the viscosity and/or density of the composition. For example, the composition comprises a contrast agent to increase the viscosity and/or density of the composition. Exemplary compositions comprise about 20 to 40% non-ionic, low-osmolar compound or contrast agent or about 25% to about 35% non-ionic, low-osmolar compound. An exemplary composition comprises scAAV or rAAV viral particles formulated in 20 mM Tris (pH8.0), 1 mM MgCl2, 200 mM NaCl, 0.001% poloxamer 188 and about 25% to about 35% non-ionic, low-osmolar compound. Another exemplary composition comprises scAAV formulated in and 1X PBS and 0.001% Pluronic F68.IG.

Methods of transducing target cells (including, but not limited to, a cell of the nervous system, e.g., nerve or glial cells) with rAAV are provided. The cells of the nervous system, including neurons, lower motor neurons, microglial cells, oligodendrocytes, astrocytes, Schwann cells or combinations thereof.

The term “transduction” is used to refer to the administration/delivery of the CLN1 polynucleotide to a target cell either in vivo or in vitro, via a replication-deficient rAAV of the disclosure resulting in expression of a functional polypeptide by the recipient cell. Transduction of cells with rAAV of the disclosure results in sustained expression of polypeptide or RNA encoded by the rAAV. The present disclosure thus provides methods of administering/delivering to a subject rAAV encoding a CLN1 polypeptide by an intrathecal, intracerebroventricular, intraparechymal, or intravenous route, or any combination thereof. Intrathecal delivery refers to delivery into the space under the arachnoid membrane of the brain or spinal cord. In some embodiments, intrathecal administration is via intracisternal (i.e. into the cerebrospinal fluid) administration.

In some embodiments of treatment methods of the invention, an agent that increases viscosity and/or density of the composition is administered to the patient. For example, a non-ionic, low-osmolar contrast agent is also administered to the patient. Such contrast agents include, but are not limited to, iobitridol, iohexol, iomeprol, iopamidol, iopentol, iopromide, ioversol, ioxilan, and mixtures of two or more of the contrast agents. In some embodiments, the treatment methods thus further comprise administration of iohexol to the patient. The non-ionic, low-osmolar contrast agent is contemplated to increase transduction of target cells in the central nervous system of the patient. It is contemplated that the transduction of cells is increased when a rAAV of the disclosure is used in combination with a contrast agent as described herein relative to the transduction of cells when a rAAV of the disclosure is used alone. In various embodiments, the transduction of cells is increased by at least about 1%, or at least about 5%, at least about 10%, at least about 20%, at least about 30%, at least about 40%, at least about 50%, at least about 60%, at least about 70%, at least about 80%, at least about 90%, at least about 100%, at least about 120%, at least about 150%, at least about 180%, at least about 200%, at least about 250%, at least about 300%, at least about 350%, at least about 400%, at least about 450%, at least about 500% or more when a vector of the disclosure is used in combination with a contrast agent as described herein, relative to the transduction of a vector of the disclosure when not used in combination with a contrast agent. In further embodiments, the transduction of cells is increased by about 10% to about 50%, or by about 10% to about 100%, or by about 5% to about 10%, or by about 5% to about 50%, or by about 1% to about 500%, or by about 10% to about 200%, or by about 10% to about 300%, or by about 10% to about 400%, or by about 100% to about 500%, or by about 150% to about 300%, or by about 200% to about 500% when a vector of the disclosure is used in combination with a contrast agent as described herein, relative to the transduction of a vector of the disclosure when not used in combination with a contrast agent.

In some embodiments of methods provided herein, the patient is held in the Trendelenberg position (head down position) after administration of the rAAV (e.g., for about 5, about 10, about 15 or about 20 minutes). In some embodiments, for example, the patients is tilted in the head down position at about 1 degree to about 30 degrees, about 15 to about 30 degrees, about 30 to about 60 degrees, about 60 to about 90 degrees, or about 90 up to about 180 degrees) during or after intrathecal vector infusion. In various embodiments, the transduction of cells is increased by at least about 1%, or at least about 5%, at least about 10%, at least about 20%, at least about 30%, at least about 40%, at least about 50%, at least about 60%, at least about 70%, at least about 80%, at least about 90%, at least about 100%, at least about 120%, at least about 150%, at least about 180%, at least about 200%, at least about 250%, at least about 300%, at least about 350%, at least about 400%, at least about 450%, at least about 500% or more when a the Trendelenberg position is used as described herein, relative to when the Trendelenberg position is not used.

In further embodiments, the transduction of cells is increased by about 10% to about 50%, or by about 10% to about 100%, or by about 5% to about 10%, or by about 5% to about 50%, or by about 1% to about 500%, or by about 10% to about 200%, or by about 10% to about 300%, or by about 10% to about 400%, or by about 100% to about 500%, or by about 150% to about 300%, or by about 200% to about 500% when a vector of the disclosure is used in combination with a contrast agent and the Trendelenberg position as described herein, relative to the transduction of a vector of the disclosure when not used in combination with a contrast agent and Trendelenberg position.

The disclosure also provides treatment method embodiments wherein the intrathecal administration of a vector of the disclosure and a contrast agent to the central nervous system of a patient in need thereof who is put in the Trendelenberg position results in a further increase in survival of the patient relative to survival of the patient when a vector of the disclosure is administered in the absence of the contrast agent and the Trendelenberg position. In various embodiments, administration of a vector of the disclosure and a contrast agent to the central nervous system of a patient in need thereof put in the Trendelenberg position results in an increase of survival of the patient of at least about 1%, at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more relative to survival of the patient when a vector of the disclosure is administered in the absence of the contrast agent and the Trendelenberg position.

Intrathecal administration is exemplified herein. These methods include transducing target cells (including, but not limited to, nerve and/or glial cells) with one or more rAAV described herein. In some embodiments, the rAAV viral particle comprising a polynucleotide encoding a CLN1 polypeptide is administered or delivered the brain and/or spinal cord of a patient. In some embodiments, the polynucleotide is delivered to brain. Areas of the brain contemplated for delivery include, but are not limited to, the motor cortex and the brain stem. In some embodiments, the polynucleotide is delivered to the spinal cord. In some embodiments, the polynucleotide is delivered to a neuron or lower motor neuron. In some embodiments, the polynucleotide is delivered to nerve and glial cells. In some embodiments, the glial cell is a microglial cell, an oligodendrocyte or an astrocyte. In some embodiments, the polynucleotide is delivered to a Schwann cell.

“Treatment” comprises the step of administering, such as administering via the intrathecal route or intravenous route, an effective dose, or effective multiple doses, of a composition comprising a rAAV of the invention to a subject animal (including a human patient) in need thereof.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. The methods provided herein comprise the step of administering an effective dose, or effective multiple doses, of a composition comprising a rAAV provided herein to a subject (e.g., an animal including, but not limited to, a human patient) in need thereof. If the dose is administered prior to development of CLN1-Batten Disease, the administration is prophylactic. If the dose is administered after the development of CLN1-Batten Disease, the administration is therapeutic. An effective dose is a dose that alleviates (eliminates or reduces) at least one symptom associated with the disease, that slows or prevents progression of the disease, that diminishes the extent of disease, that results in remission (partial or total) of disease, and/or that prolongs survival. In various embodiments, the dose of a composition comprising a rAAV disclosed herein for use in the methods provided is based on the size and growth rate of the nervous system and the cerebrospinal fluid. The dose is not based on the body weight but on absolute dose per patient. The dosing regimen is based on the fact that the size of the nervous system and the volume of the cerebrospinal fluid does not differ as much between individuals compared to the body weight that can be vastly different.

In comparison to the subject before treatment or in comparison to an untreated subject, methods provided herein result in stabilization, reduced progression, or improvement in one or more of the scales that are used to evaluate progression and/or improvement in CLN1-Batten-disease, e.g. the Unified Batten Disease Rating System (UBDRS) assessment scales or the Hamburg Motor and Language Scale. The UBDRS assessment scales (as described in Marshall et al., Neurology. 2005 65 (2): 275-279) [including the UBDRS physical assessment scale, the UBDRS seizure assessment scale, the UBDRS behavioral assessment scale, the UBDRS capability assessment scale, the UBDRS sequence of symptom onset, and the UBDRS Clinical Global Impressions (CGI)]; the Pediatric Quality of Life Scale (PEDSQOL) scale, motor function, language function, cognitive function, and survival.

In comparison to the subject before treatment or in comparison to an untreated subject, methods provided herein result in one or more of the following: reduced or slowed lysosomal accumulation of autofluorescent storage material, reduced or slowed lysosomal accumulation of ATP Synthase Subunit C, reduced or slowed glial activation (astrocytes and/or microglia) activation; reduced or slowed astrocytosis, and reduction in brain volume loss or slowing of brain volume loss measured by MRI, reduced or slowed onset of seizures, and stabilization, reduced or slowed progression, or improvement in one or more of the UBDRS assessment scales or Hamburg Motor and Language Scale, wherein the reduction, stabilization, or improvement is as compared to the patient prior to treatment or to an untreated CLN1-Batten Disease patient.

Combination therapies are also provided. Combination as used herein includes either simultaneous treatment or sequential treatment. Combinations of methods described herein with standard medical treatments are specifically contemplated, as are combinations with novel therapies. In some embodiments, the combination therapy comprises administering an immunosuppressing agent in combination with the gene therapy disclosed herein.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the disease state being treated and the target cells/tissue(s) that are to express the CLN1 protein.

The disclosure provides for local administration and systemic administration of an effective dose of rAAV and compositions of the disclosure. For example, systemic administration is administration into the circulatory system so that the entire body is affected.

While delivery to a subject in need thereof after birth is contemplated, intrauterine delivery to a fetus is also contemplated.

### Immunosuppressing Agents

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, hydrocortisone, methylprednisolone, deflazacort, budesonide or prednisone. Janus kinase inhibitors are inhibitors of the JAK/STAT signaling pathway by targeting one or more of the Janus kinase family of enzymes. Exemplary janus kinase inhibitors include tofa-citinib, baricitinib, upadacitinib, peficitinib, and oclacitinib.

Calcineurin inhibitors bind to cyclophilin and inhibits the activity of calcineurin Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include sirolimus, everolimus, and temsirolimus.

The immunosuppressing agents include immune suppressing macrolides. The term “immune suppressing macrolides” refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and sirolimus.

Purine analogs block nucleotide synthesis and include IMDH inhibitors. Exemplary purine analogs include azathioprine, mycophenolate and lefunomide.

Exemplary immunosuppressing biologics include abatacept, adalimumab, anakinra, certoli-zumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immuosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

### EXAMPLES

While the following examples describe specific embodiments, it is understood that variations and modifications will occur to those skilled in the art. Accordingly, only such limitations as appear in the claims should be placed on the invention.

In the examples, a self-complementary AAV carrying a CLN1 cDNA under the control of a P546 promoter (named scAAV9.P546.CLN1) or a cmv-enhancer chicken β-actin promoter (named scAAV9.CB.CLN1) was produced. The P546 promoter is a truncated version of the Methyl CpG binding protein 2 (MeCP2) promoter, allowing expression of the transgene in both neurons and astrocytes at moderate levels.

### Example 1

### Production of scAAV9.P546.CLN1

A cDNA expression clone of human CLN1 was obtained from Origene (SC119961). The DNA sequence including the open reading frame of human CLN1 (SEQ ID NO: 1) was amplified from this plasmid and then inserted in a double-stranded AAV2-ITR-based production plasmid between Agel and Sbfl restriction endonuclease sites. A schematic of the plasmid construct showing the CLN1 DNA inserted between AAV2 ITRs (the 5′ ITR was modified as previously described in McCarty et al., Gene Therapy 8:1248-1254 (2001) to generate scAAV) is shown in FIG. 1. The plasmid construct also includes the P546 promoter, an SV40 chimeric intron and a Bovine Growth Hormone (BGH) polyadenylation signal.

scAAV9.P546.CLN1 scAAV was produced under cGMP conditions by transient triple-plasmid transfection procedures using the double-stranded AAV2-ITR-based production plasmid, with a plasmid encoding Rep2Cap9 sequence as previously described [Gao et al., J. Virol., 78:6381-6388 (2004)], along with an adenoviral helper plasmid pHelper (Stratagene, Santa Clara, CA) in HEK293 cells. Virus was purified by two cesium chloride density gradient purification steps, dialyzed against PBS and formulated with 0.001% Pluronic-F68 to prevent virus aggregation and stored at 4° C. All scAAV preparations were titered by quantitative PCR using Taq-Man technology. Purity of scAAV was assessed by 4-12% sodium dodecyl sulfate-acrylamide gel electrophoresis and silver staining (Invitrogen, Carlsbad, CA).

### Example 2

### Production of scAAV9.CB.CLN1

A cDNA expression clone of human CLN1 was obtained from Origene (SC119961). The DNA sequence including the open reading frame of human CLN1 (SEQ ID NO: 1) was amplified from this plasmid and then inserted in a double-stranded AAV2-ITR-based production plasmid between Agel and Sbf1 restriction endonuclease sites. A schematic of the plasmid construct showing the CLN1 DNA inserted between AAV2 ITRs (the 5′ ITR was modified as previously described in McCarty et al., Gene Therapy 8:1248-1254 (2001) to generate scAAV) is shown in FIG. 3. The plasmid construct also includes a CMV enhancer, a CB promoter (labelled CBA promoter), an SV40 chimeric intron and a Bovine Growth Hormone (BGH) polyadenylation signal.

scAAV9.CB.CLN1 scAAV was produced under cGMP conditions by transient triple-plasmid transfection procedures using the double-stranded AAV2-ITR-based production plasmid, with a plasmid encoding Rep2Cap9 sequence as previously described [Gao et al., J. Virol., 78:6381-6388 (2004)], along with an adenoviral helper plasmid pHelper (Stratagene, Santa Clara, CA) in HEK293 cells. Virus was purified by two cesium chloride density gradient purification steps, dialyzed against PBS and formulated with 0.001% Pluronic-F68 to prevent virus aggregation and stored at 4° C. All scAAV preparations were titered by quantitative PCR using Taq-Man technology. Purity of scAAV was assessed by 4-12% sodium dodecyl sulfate-acrylamide gel electrophoresis and silver staining (Invitrogen, Carlsbad, CA).

### Example 3

### Analysis of Expression of CSF-delivered scAA V9.P546.CLN1 scAAV or scAA V9.CB.CLN1 scAAV in PPT-1 deficient mice

### Cell Targeting and Expression

To confirm the expression and biodistribution of virally-introduced human CLN1, scAAV9.P546.CLN1 scAAV or scAAV9.CB.CLN1 scAAV is administered into PPT-1 deficient mice via intracerebroventricular (ICV) injection within 24 hours after birth and expression is monitored at various time points over a course of two months. Wild type and PPT-1 deficient mice are injected with an equal volume of PBS served as controls.

To obtain a detailed brain biodistribution profile, RNAscope in situ hybridization techniques are used to specifically identify human CLN1 mRNA in seven specific brain regions: cerebral cortex (motor (A), somatosensory (B), visual (C)), thalamus (D), hindbrain (E), cerebellum (F), and spinal cord (G)). This technique involves RNA in situ hybridization with specific probes to detect only the human transgene encoded by the AAV9. The analysis will demonstrate expression of CLN1 transgene in various regions of the brain including cortex, thalamus, hindbrain, cerebellum and spinal cord.

### Accumulation of Autofluorescent Storage Material (ASM)

Accumulation of autofluorescent storage material (ASM) is the hallmark histological marker for Batten disease progression (Mole et al., Biochim Biophys Acta-Mol Basis Dis. 2015;1852 (10): 2237-2241; Cotman et al., Clin Lipidol. 2012 February;7 (1): 79-91; Seehafer et al., Neurobiol Aging. 2006; 27:576-588). Accumulation of ASM is a strong indicator for disease progression for many forms of Batten disease (Bosch et al., J Neurosci. 2016; 36 (37): 9669-9682; Morgan et al., PLOS One. 2013; 8 (11): e78694). It is contemplated herein that reduction of ASM is used as indicator of successful treatment. Automated quantification of fluorescent pixel area confirms a significant reduction in accumulated ASM in the motor cortex, somatosensory cortex, visual cortex and thalamus in gene therapy treated PPT-1 deficient mice at 2 and 8 months of age compared to the PBS injected PPT-1 deficient mice.

### Accumulation of Mitochondrial Protein ATP Synthase Subunit C

Accumulation of ATP synthase subunit C is analyzed in brain tissue from wild type, PBS-injected gene therapy treated PPT-1 deficient mice. In healthy individuals, this protein is part of the respiratory chain in the mitochondrial membrane, but in patients suffering from Batten disease, the protein aberrantly accumulates in lysosomes (Palmer et al., Am J Med Genet. 1992;42 (4): 561-567).

### Functional Assays

In an efficacy study for following delivery of scAAV9.P546.CLN1 scAAV or scAAV9.CB.CLN1 scAAV in PPT-1 deficient mice, starting at 2 months of age, and continuing at 2-month intervals, the treated and control mice are subjected to a battery of behavioral testing paradigms including: accelerating rotarod assays, and pole climbing to test motor function and coordination, as well as Morris water maze to assess learning and memory.

The rotarod assay is performed every 2 months. Mice are placed on an accelerating wheel and time until they fall is measured. At each time point, mice are trained in the morning and testing is performed 4 hours later in the afternoon.

The pole climbing test measures the time the mice take to turn around on a vertical pole when placed on it facing upwards, as well as the time to descend the pole when placed on it facing downwards. Moreover, the number of falls from the pole while attempting to turn or descend is also measured. This test evaluates coordination and balancing capabilities.

In the Morris Water Maze test, animals are placed in a water-filled pool containing a hidden platform. After training, the time it took the animals to find the hidden platform using environmental cues for orientation is measured as a sign of learning and memory capabilities.

### Example 4

### Clinical trial of scAAV9.P546.CLN1 gene therapy

The scAAV9.P546.CLN1 is delivered intrathecally to human patients with CLN1-Batten Disease.

The scAAV for the clinical trial is produced by the Nationwide Children's Hospital Clinical Manufacturing Facility utilizing a triple-transfection method of HEK293 cells, under cGMP conditions as described in Example 1.

Patients selected for participation have a diagnosis of CLN1 disease as determined by genotype. The first cohort (n=3) receive a one- or two-time gene transfer a dose viral vector per patient of between 1×10¹³ and 1×10¹⁵ vg per patient. The scAAV9.P546.CLN1 is formulated 20 mM Tris (pH8.0), 1 mM MgCl2, 200 mM NaCl, 0.001%. poloxamer 188 and about 20% to about 40% non-ionic, low-osmolar compound delivered one-time through an intrathecal catheter inserted by a lumbar puncture into the interspinous into the subarachnoid space of the lumbar thecal sac. Safety is assessed on clinical grounds, and by examination of safety labels. There is a minimum of four weeks between enrollments of each subject to allow for review of Day 30 post-gene transfer safety data. If there are no safety concerns, after the third subject is evaluated at one month post-injection, a second cohort of four additional subjects is enrolled. Each subject in cohort 2 (n=4) receives an escalated dose of viral vector. There is at least a six week window between the completion of Cohort 1 and the start of Cohort 2, to allow review of the safety analysis from five time points (days 1, 2, 7, 14, and 21) as well as DSMB review prior to dosing of the next subject.

Disease progression is measured with the UBDRS scales or the Hamburg Motor and Language Scale (referenced in the Detailed Description above) and the impact of treatment on quality of life using the Pediatric Quality of Life (PEDSQOL) scale, and potential for prolonged survival.

The primary analysis for efficacy is assessed when all patients have completed the three-year study. Basis of determining efficacy is by stabilization or reduced progression of the disease based on the well-established Unified Batten Disease Rating Scale (UBDRS) that was developed specifically for CLN1-Batten Disease or the Hamburg Motor and Language Scale. Upon completion of the three-year study period patients are monitored annually for 5 years as per FDA guidance.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

All documents referred to in this application are hereby incorporated by reference in their entirety.

SEQUENCES

SEQ ID NO: 1-CLN1 nucleotide
atggcgtcgcccggctgcctgtggctcttggctgtggctctcctgccatggacctgcgcttctcgggcgctgcagcatctggacccgccgg
cgccgctgccgttggtgatctggcatgggatgggagacagctgttgcaatcccttaagcatgggtgctattaaaaaaatggtggagaagaa
aatacctggaatttacgtcttatctttagagattgggaagaccctgatggaggacgtggagaacagcttcttcttgaatgtcaattcccaa
gtaacaacagtgtgtcaggcacttgctaaggatcctaaattgcagcaaggctacaatgctatgggattctcccagggaggccaatttctga
gggcagtggctcagagatgcccttcacctcccatgatcaatctgatctcggttgggggacaacatcaaggtgtttttggactccctcgatg
cccaggagagagctctcacatctgtgacttcatccgaaaaacactgaatgctggggcgtactccaaagttgttcaggaacgcctcgtgcaa
gccgaatactggcatgaccccataaaggaggatgtgtatcgcaaccacagcatcttcttggcagatataaatcaggagcggggtatcaatg
agtcctacaagaaaaacctgatggccctgaagaagtttgtgatggtgaaattcctcaatgattccattgtggaccctgtagattcggagtg
gtttggattttacagaagtggccaagccaaggaaaccattcccttacaggagacctccctgtacacacaggaccgcctggggctaaaggaa
atggacaatgcaggacagctagtgtttctggctacagaaggggaccatcttcagttgtctgaagaatggttttatgcccacatcataccat
tccttggatga
SEQ ID NO: 2-CLN1 polypeptide
MASPGCLWLLAVALLPWTCASRALQHLDPPAPLPLVIWHGMGDSCCNPLSMGAIKKMVEK
KIPGIYVLSLEIGKTLMEDVENSFFLNVNSQVTTVCQALAKDPKLQQGYNAMGFSQGGQFLR
AVAQRCPSPPMINLISVGGQHQGVFGLPRCPGESSHICDFIRKTLNAGAYSKVVQERLVQAEY
WHDPIKEDVYRNHSIFLADINQERGINESYKKNLMALKKFVMVKFLNDSIVDPVDSEWFGFY
RSGQAKETIPLQETSLYTQDRLGLKEMDNAGQLVFLATEGDHLQLSEEWFYAHIIPFLG
SEQ ID NO: 3-P456 promoter
gaacaacgccaggctcctcaacaggcaactttgctacttctacagaaaatgataataaagaaatgctggtgaagtcaaatgcttatcacaa
tggtgaactactcagcagggaggctctaataggcgccaagagcctagacttccttaagcgccagagtccacaagggcccagttaatcctca
acattcaaatgctgcccacaaaaccagcccctctgtgccctagccgcctcttttttccaagtgacagtagaactccaccaatccgcagctg
aatggggtccgcctcttttccctgcctaaacagacaggaactcctgccaattgagggcgtcaccgctaaggctccgccccagcctgggctc
cacaaccaatgaagggtaatctcgacaaagagcaaggggtggggcgcgggcgcgcaggtgcagcagcacacaggctggtcgggagggcggg
gcgcgacgtctgccgtgcggggtcccggcatcggttgcgcgcgcgctccctcctctcggagagagggctgtggtaaaacccgtccggaaaa
SEQ ID NO: 4-Hybrid chicken B-Actin (CB) promoter
ccacgttctgcttcactctccccatctcccccccctccccacccccaattttgtatttatttattttttaattattttgtgcagcgatggg
ggcggggggggggggggggcgcgcgccaggcggggcggggcggggcgaggggcggggcggggcgaggcggagaggtgcggcggcagccaat
cagagcggcgcgctccgaaagtttccttttatggcgaggcggcggcggcggcggccctataaaaagcgaagcgcgcggcgggcgggag
SEQ ID NO: 5-pscAAV9.P546.CLN1.Kan vector
gcccaatacgcaaaccgcctctccccgcgcgttggccgattcattaatgcagctggcgtaatagcgaagaggcccgcaccgatcgcccttc
ccaacagttgcgcagcctgaatggcgaatggcgattccgttgcaatggctggcggtaatattgttctggatattaccagcaaggccgatag
tttgagttcttctactcaggcaagtgatgttattactaatcaaagaagtattgcgacaacggttaatttgcgtgatggacagactctttta
ctcggtggcctcactgattataaaaacacttctcaggattctggcgtaccgttcctgtctaaaatccctttaatcggcctcctgtttagct
cccgctctgattctaacgaggaaagcacgttatacgtgctcgtcaaagcaaccatagtacgcgccctgtagcggcgcattaagcgcggcgg
gtgtggtggttacgcgcagcgtgaccgctacacttgccagcgccctagcgcccgctcctttcgctttcttcccttcctttctcgccacgtt
cgccggctttccccgtcaagctctaaatcgggggctccctttagggttccgatttagtgctttacggcacctcgaccccaaaaaacttgat
tagggtgatggttcacgtagtgggccatcgccctgatagacggtttttcgccctttgacgttggagtccacgttctttaatagtggactct
tgttccaaactggaacaacactcaaccctatctcggtctattcttttgatttataagggattttgccgatttcggcctattggttaaaaaa
tgagctgatttaacaaaaatttaacgcgaattttaacaaaatattaacgcttacaatttaaatatttgcttatacaatcttcctgtttttg
gggcttttctgattatcaaccggggtacatatgattgacatgctagttttacgattaccgttcatcgccctgcgcgctcgctcgctcactg
aggccgcccgggcaaagcccgggcgtcgggcgacctttggtcgcccggcctcagtgagcgagcgagcgcgcagagagggagtggaattcaa
ttcacgcgccggtaccgaattcacgcgtgaacaacgccaggctcctcaacaggcaactttgctacttctacagaaaatgataataaagaaa
tgctggtgaagtcaaatgcttatcacaatggtgaactactcagcagggaggctctaataggcgccaagagcctagacttccttaagcgcca
gagtccacaagggcccagttaatcctcaacattcaaatgctgcccacaaaaccagcccctctgtgccctagccgcctcttttttccaagtg
acagtagaactccaccaatccgcagctgaatggggtccgcctcttttccctgcctaaacagacaggaactcctgccaattgagggcgtcac
cgctaaggctccgccccagcctgggctccacaaccaatgaagggtaatctcgacaaagagcaaggggtggggcgcgggcgcgcaggtgcag
cagcacacaggctggtcgggagggcggggcgcgacgtctgccgtgcggggtcccggcatcggttgcgcgcgcgctccctcctctcggagag
agggctgtggtaaaacccgtccggaaaacgcgtcgaagggcgaattctgcagataactggtaagtttagtcttttttgtcttttatttcag
gtcccggatccggtggtggtgcaaatcaaagaactgctcctcagtcgatgttgcctttacttctaggcctgtacggaagtgttactaccgg
tATGGCGTCGCCCGGCTGCCTGTGGCTCTTGGCTGTGGCTCTCCTGCCATGGACCTGCGCTTCT
CGGGCGCTGCAGCATCTGGACCCGCCGGCGCCGCTGCCGTTGGTGATCTGGCATGGGATGGGAGA
CAGCTGTTGCAATCCCTTAAGCATGGGTGCTATTAAAAAAATGGTGGAGAAGAAAATACCTGGA
ATTTACGTCTTATCTTTAGAGATTGGGAAGACCCTGATGGAGGACGTGGAGAACAGCTTCTTCTT
GAATGTCAATTCCCAAGTAACAACAGTGTGTCAGGCACTTGCTAAGGATCCTAAATTGCAGCAAG
GCTACAATGCTATGGGATTCTCCCAGGGAGGCCAATTTCTGAGGGCAGTGGCTCAGAGATGCC
CTTCACCTCCCATGATCAATCTGATCTCGGTTGGGGGACAACATCAAGGTGTTTTTGGAC
TCCCTCGATGCCCAGGAGAGAGCTCTCACATCTGTGACTTCATCCGAAAAACACTGAATG
CTGGGGCGTACTCCAAAGTTGTTCAGGAACGCCTCGTGCAAGCCGAATACTGGCATGAC
CCCATAAAGGAGGATGTGTATCGCAACCACAGCATCTTCTTGGCAGATATAAATCAGGA
GCGGGGTATCAATGAGTCCTACAAGAAAAACCTGATGGCCCTGAAGAAGTTTGTGATGG
TGAAATTCCTCAATGATTCCATTGTGGACCCTGTAGATTCGGAGTGGTTTGGATTTTACA
GAAGTGGCCAAGCCAAGGAAACCATTCCCTTACAGGAGACCTCCCTGTACACACAGGAC
CGCCTGGGGCTAAAGGAAATGGACAATGCAGGACAGCTAGTGTTTCTGGCTACAGAAGG
GGACCATCTTCAGTTGTCTGAAGAATGGTTTTATGCCCACATCATACCATTCCTTGGATG
Acctgcaggcctcgactgtgccttctagttgccagccatctgttgtttgcccctcccccgtgccttccttgaccctggaaggtgccactcc
cactgtcctttcctaataaaatgaggaaattgcatcgcattgtctgagtaggtgtcattctattcgcatgctggggagagatcgatctgag
gaacccctagtgatggagttggccactccctctctgcgcgctcgctcgctcactgaggccgggcgaccaaaggtcgcccgacgcccgggct
ttgcccgggcggcctcagtgagcgagcgagcgcgcagagagggagtggcccccccccccccccccccggcgattctcttgtttgctccaga
ctctcaggcaatgacctgatagcctttgtagagacctctcaaaaatagctaccctctccggcatgaatttatcagctagaacggttgaata
tcatattgatggtgatttgactgtctccggcctttctcacccgtttgaatctttacctacacattactcaggcattgcatttaaaatatat
gagggttctaaaaatttttatccttgcgttgaaataaaggcttctcccgcaaaagtattacagggtcataatgtttttggtacaaccgatt
tagctttatgctctgaggctttattgcttaattttgctaattctttgccttgcctgtatgatttattggatgttggaatcgcctgatgcgg
tattttctccttacgcatctgtgcggtatttcacaccgcatatggtgcactctcagtacaatctgctctgatgccgcatagttaagccagc
cccgacacccgccaacactatggtgcactctcagtacaatctgctctgatgccgcatagttaagccagccccgacacccgccaacacccgc
tgacgcgccctgacgggcttgtctgctcccggcatccgcttacagacaagctgtgaccgtctccgggagctgcatgtgtcagaggttttca
ccgtcatcaccgaaacgcgcgagacgaaagggcctcgtgatacgcctatttttataggttaatgtcatgagacaataaccctgataaatgc
ttcaataatattgaaaaaggaagagtatgagccatattcaacgggaaacgtcgaggccgcgattaaattccaacatggatgctgatttata
tgggtataaatgggctcgcgataatgtcgggcaatcaggtgcgacaatctatcgcttgtatgggaagcccgatgcgccagagttgtttctg
aaacatggcaaaggtagcgttgccaatgatgttacagatgagatggtcagactaaactggctgacggaatttatgccacttccgaccatca
agcattttatccgtactcctgatgatgcatggttactcaccactgcgatccccggaaaaacagcgttccaggtattagaagaatatcctga
ttcaggtgaaaatattgttgatgcgctggcagtgttcctgcgccggttgcactcgattcctgtttgtaattgtccttttaacagcgatcgc
gtatttcgcctcgctcaggcgcaatcacgaatgaataacggtttggttgatgcgagtgattttgatgacgagcgtaatggctggcctgttg
aacaagtctggaaagaaatgcataaacttttgccattctcaccggattcagtcgtcactcatggtgatttctcacttgataaccttatttt
tgacgaggggaaattaataggttgtattgatgttggacgagtcggaatcgcagaccgataccaggatcttgccatcctatggaactgcctc
ggtgagttttctccttcattacagaaacggctttttcaaaaatatggtattgataatcctgatatgaataaattgcagtttcatttgatgc
tcgatgagtttttctaactgtcagaccaagtttactcatatatactttagattgatttaaaacttcatttttaatttaaaaggatctaggt
gaagatcctttttgataatctcatgaccaaaatcccttaacgtgagttttcgttccactgagcgtcagaccccgtagaaaagatcaaagga
tcttcttgagatcctttttttctgcgcgtaatctgctgcttgcaaacaaaaaaaccaccgctaccagcggtggtttgtttgccggatcaag
agctaccaactctttttccgaaggtaactggcttcagcagagcgcagataccaaatactgttcttctagtgtagccgtagttaggccacca
cttcaagaactctgtagcaccgcctacatacctcgctctgctaatcctgttaccagtggctgctgccagtggcgataagtcgtgtcttacc
gggttggactcaagacgatagttaccggataaggcgcagcggtcgggctgaacggggggttcgtgcacacagcccagcttggagcgaacga
cctacaccgaactgagatacctacagcgtgagctatgagaaagcgccacgcttcccgaagggagaaaggcggacaggtatccggtaagcgg
cagggtcggaacaggagagcgcacgagggagcttccagggggaaacgcctggtatctttatagtcctgtcgggtttcgccacctctgactt
gagcgtcgatttttgtgatgctcgtcaggggggcggagcctatggaaaaacgccagcaacgcggcctttttacggttcctggccttttgct
ggccttttgctcacatgttctttcctgcgttatcccctgattctgtggataaccgtattaccgcctttgagtgagctgataccgctcgccg
cagccgaacgaccgagcgcagcgagtcagtgagcgaggaagcggaagagc
SEQ ID NO: 6-pscAAV9.CB.CLN1.Kan
CTGATTCTAACGAGGAAAGCACGTTATACGTGCTCGTCAAAGCAACCATAGTACGCGCC
CTGTAGCGGCGCATTAAGCGCGGCGGGTGTGGTGGTTACGCGCAGCGTGACCGCTACAC
TTGCCAGCGCCCTAGCGCCCGCTCCTTTCGCTTTCTTCCCTTCCTTTCTCGCCACGTTCGCC
GGCTTTCCCCGTCAAGCTCTAAATCGGGGGCTCCCTTTAGGGTTCCGATTTAGTGCTTTAC
GGCACCTCGACCCCAAAAAACTTGATTAGGGTGATGGTTCACGTAGTGGGCCATCGCCCT
GATAGACGGTTTTTCGCCCTTTGACGTTGGAGTCCACGTTCTTTAATAGTGGACTCTTGTT
CCAAACTGGAACAACACTCAACCCTATCTCGGTCTATTCTTTTGATTTATAAGGGATTTTG
CCGATTTCGGCCTATTGGTTAAAAAATGAGCTGATTTAACAAAAATTTAACGCGAATTTT
AACAAAATATTAACGCTTACAATTTAAATATTTGCTTATACAATCTTCCTGTTTTTGGGGC
TTTTCTGATTATCAACCGGGGTACATATGATTGACATGCTAGTTTTACGATTACCGTTCAT
CGCCCTGCGCGCTCGCTCGCTCACTGAGGCCGCCCGGGCAAAGCCCGGGCGTCGGGCGA
CCTTTGGTCGCCCGGCCTCAGTGAGCGAGCGAGCGCGCAGAGAGGGAGTGGAATTCACG
CGTGGATCTGAATTCAATTCACGCGTGGTACCTCTGGTCGTTACATAACTTACGGTAAAT
GGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTACGGTAA
ACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCT
ACTTGGCAGTACATCTACTCGAGGCCACGTTCTGCTTCACTCTCCCCATCTCCCCCCCCTC
CCCACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGGGGGCGGGGCG
AGGCGGAGAGGTGCGGCGGCAGCCAATCAGAGCGGCGCGCTCCGAAAGTTTCCTTTTAT
GGCGAGGCGGCGGCGGCGGCGGCCCTATAAAAAGCGAAGCGCGCGGCGGGCGGGAGCG
GGATCAGCCACCGCGGTGGCGGCCTAGAGTCGACGAGGAACTGAAAAACCAGAAAGTT
AACTGGTAAGTTTAGTCTTTTTGTCTTTTATTTCAGGTCCCGGATCCGGTGGTGGTGCAAA
TCAAAGAACTGCTCCTCAGTGGATGTTGCCTTTACTTCTAGGCCTGTACGGAAGTGTTAC
TTCTGCTCTAAAAGCTGCGGAATTGTACCCGCGGCCGATCCAccggtATGGCGTCGCCCGGC
TGCCTGTGGCTCTTGGCTGTGGCTCTCCTGCCATGGACCTGCGCTTCTCGGGCGCTGCAGC
ATCTGGACCCGCCGGCGCCGCTGCCGTTGGTGATCTGGCATGGGATGGGAGACAGCTGTT
GCAATCCCTTAAGCATGGGTGCTATTAAAAAAATGGTGGAGAAGAAAATACCTGGAATT
TACGTCTTATCTTTAGAGATTGGGAAGACCCTGATGGAGGACGTGGAGAACAGCTTCTTC
TTGAATGTCAATTCCCAAGTAACAACAGTGTGTCAGGCACTTGCTAAGGATCCTAAATTG
CAGCAAGGCTACAATGCTATGGGATTCTCCCAGGGAGGCCAATTTCTGAGGGCAGTGGC
TCAGAGATGCCCTTCACCTCCCATGATCAATCTGATCTCGGTTGGGGGACAACATCAAGG
TGTTTTTGGACTCCCTCGATGCCCAGGAGAGAGCTCTCACATCTGTGACTTCATCCGAAA
AACACTGAATGCTGGGGCGTACTCCAAAGTTGTTCAGGAACGCCTCGTGCAAGCCGAAT
ACTGGCATGACCCCATAAAGGAGGATGTGTATCGCAACCACAGCATCTTCTTGGCAGAT
ATAAATCAGGAGCGGGGTATCAATGAGTCCTACAAGAAAAACCTGATGGCCCTGAAGAA
GTTTGTGATGGTGAAATTCCTCAATGATTCCATTGTGGACCCTGTAGATTCGGAGTGGTTT
GGATTTTACAGAAGTGGCCAAGCCAAGGAAACCATTCCCTTACAGGAGACCTCCCTGTA
CACACAGGACCGCCTGGGGCTAAAGGAAATGGACAATGCAGGACAGCTAGTGTTTCTGG
CTACAGAAGGGGACCATCTTCAGTTGTCTGAAGAATGGTTTTATGCCCACATCATACCAT
TCCTTGGATGAcctgcaggcctcgactgtgccttctagttgccagccatctgttgtttgcccctcccccgtgccttccttgaccctggaag
gtgccactcccactgtcctttcctaataaaatgaggaaattgcatcgcattgtctgagtaggtgtcattctattcgcatgCTGGGGAGAGA
TCGATCTGAGGAACCCCTAGTGATGGAGTTGGCCACTCCCTCTCTGCGCGCTCGCTCGCTCA
CTGAGGCCGGGCGACCAAAGGTCGCCCGACGCCCGGGCTTTGCCCGGGCGGCCTCAGTG
AGCGAGCGAGCGCGCAGAGAGGGAGTGGCCCCCCCCCCCCCCCCCCCGGCGATTCTCTT
GTTTGCTCCAGACTCTCAGGCAATGACCTGATAGCCTTTGTAGAGACCTCTCAAAAATAG
CTACCCTCTCCGGCATGAATTTATCAGCTAGAACGGTTGAATATCATATTGATGGTGATT
TGACTGTCTCCGGCCTTTCTCACCCGTTTGAATCTTTACCTACACATTACTCAGGCATTGC
ATTTAAAATATATGAGGGTTCTAAAAATTTTTATCCTTGCGTTGAAATAAAGGCTTCTCCC
GCAAAAGTATTACAGGGTCATAATGTTTTTGGTACAACCGATTTAGCTTTATGCTCTGAG
GCTTTATTGCTTAATTTTGCTAATTCTTTGCCTTGCCTGTATGATTTATTGGATGTTGGAAT
CGCCTGATGCGGTATTTTCTCCTTACGCATCTGTGCGGTATTTCACACCGCATATGGTGCA
CTCTCAGTACAATCTGCTCTGATGCCGCATAGTTAAGCCAGCCCCGACACCCGCCAACAC
TATGGTGCACTCTCAGTACAATCTGCTCTGATGCCGCATAGTTAAGCCAGCCCCGACACC
CGCCAACACCCGCTGACGCGCCCTGACGGGCTTGTCTGCTCCCGGCATCCGCTTACAGAC
AAGCTGTGACCGTCTCCGGGAGCTGCATGTGTCAGAGGTTTTCACCGTCATCACCGAAAC
GCGCGAGACGAAAGGGCCTCGTGATACGCCTATTTTTATAGGTTAATGTCATGAgattatcaaa
aaggatcttcacctagatccttttaaattaaaaatgaagttttaaatcaatctaaagtatatatgagtaaacttggtctgacagttagaaa
aactcatcgagcatcaaatgaaactgcaatttattcatatcaggattatcaataccatatttttgaaaaagccgtttctgtaatgaaggag
aaaactcaccgaggcagttccataggatggcaagatcctggtatcggtctgcgattccgactcgtccaacatcaatacaacctattaattt
cccctcgtcaaaaataaggttatcaagtgagaaatcaccatgagtgacgactgaatccggtgagaatggcaaaagtttatgcatttctttc
cagacttgttcaacaggccagccattacgctcgtcatcaaaatcactcgcatcaaccaaaccgttattcattcgtgattgcgcctgagcga
ggcgaaatacgcgatcgctgttaaaaggacaattacaaacaggaatcgagtgcaaccggcgcaggaacactgccagcgcatcaacaatatt
ttcacctgaatcaggatattcttctaatacctggaacgctgtttttccggggatcgcagtggtgagtaaccatgcatcatcaggagtacgg
ataaaatgcttgatggtcggaagtggcataaattccgtcagccagtttagtctgaccatctcatctgtaacatcattggcaacgctacctt
tgccatgtttcagaaacaactctggcgcatcgggcttcccatacaagcgatagattgtcgcacctgattgcccgacattatcgcgagccca
tttatacccatataaatcagcatccatgttggaatttaatcgcggcctcgacgtttcccgttgaatatggctcatactcttcctttttcaa
tattattgaagcatttatcagggttattgtcTCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAAAAAAACCACCGCTACCAGCGGTGGTTTGTTT
GCCGGATCAAGAGCTACCAACTCTTTTTCCGAAGGTAACTGGCTTCAGCAGAGCGCAGATA
CCAAATACTGTTCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATAAGTCGT
GTCTTACCGGGTTGGACTCAAGACGATAGTTACCGGATAAGGCGCAGCGGTCGGGCTGA
ACGGGGGGTTCGTGCACACAGCCCAGCTTGGAGCGAACGACCTACACCGAACTGAGATA
CCTACAGCGTGAGCTATGAGAAAGCGCCACGCTTCCCGAAGGGAGAAAGGCGGACAGGT
ATCCGGTAAGCGGCAGGGTCGGAACAGGAGAGCGCACGAGGGAGCTTCCAGGGGGAAA
CGCCTGGTATCTTTATAGTCCTGTCGGGTTTCGCCACCTCTGACTTGAGCGTCGATTTTTG
TGATGCTCGTCAGGGGGGCGGAGCCTATGGAAAAACGCCAGCAACGCGGCCTTTTTACG
GTTCCTGGCCTTTTGCTGGCCTTTTGCTCACATGTTCTTTCCTGCGTTATCCCCTGATTCTG
TGGATAACCGTATTACCGCCTTTGAGTGAGCTGATACCGCTCGCCGCAGCCGAACGACCG
AGCGCAGCGAGTCAGTGAGCGAGGAAGCGGAAGAGCGCCCAATACGCAAACCGCCTCT
CCCCGCGCGTTGGCCGATTCATTAATGCAGCTGGCGTAATAGCGAAGAGGCCCGCACCG
ATCGCCCTTCCCAACAGTTGCGCAGCCTGAATGGCGAATGGCGATTCCGTTGCAATGGCT
GGCGGTAATATTGTTCTGGATATTACCAGCAAGGCCGATAGTTTGAGTTCTTCTACTCAG
GCAAGTGATGTTATTACTAATCAAAGAAGTATTGCGACAACGGTTAATTTGCGTGATGGA
CAGACTCTTTTACTCGGTGGCCTCACTGATTATAAAAACACTTCTCAGGATTCTGGCGTA
CCGTTCCTGTCTAAAATCCCTTTAATCGGCCTCCTGTTTAGCTCCCGCT

What is claimed is:

1. A polynucleotide comprising a nucleic acid sequence encoding the CLN1 polypeptide.

2. The polynucleotide of claim 1 wherein the CLN1 polypeptide comprises an amino acid sequence at least 90% identical to SEQ ID NO: 2.

3. The polynucleotide of claim 1 wherein the CLN1 polypeptide comprises the amino acid sequence of SEQ ID NO: 2.

4. The polynucleotide of any one of claims 1-3 wherein the polynucleotide sequence encoding the CLN1 polypeptide comprises a nucleotide sequence that is at least 90% identical to the nucleotide sequence of SEQ ID NO: 1.

5. The polynucleotide of claim 4 wherein the polynucleotide sequence encoding the CLN1 polypeptide comprises the nucleotide sequence of SEQ ID NO: 1.

6. The polynucleotide of any one of claims 1-5 wherein the polynucleotide further comprises the P456 promoter or the chicken β-actin (CB) promoter.

7. The polynucleotide of any one of claims 1-5 comprising a nucleotide sequence at least 90% identical to nucleotides 980-3062 of SEQ ID NO: 5.

8. The polynucleotide of any one of claims 1-5 comprising nucleotides 980-3062 of SEQ ID NO: 5.

9. The polynucleotide of any one of claims 1-5 comprising a nucleotide sequence at least 90% identical to nucleotides 610-2786 of SEQ ID NO: 6.

10. The polynucleotide of any one of claims 1-5 comprising nucleotides 610-2786 of SEQ ID NO: 6.

11. A polynucleotide comprising a P546 promoter comprising the sequence of SEQ ID NO: 3 and a nucleic acid sequence encoding the CLN1 polypeptide of SEQ ID NO: 2.

12. A polynucleotide comprising a CB promoter comprising the sequence of SEQ ID NO: 4 and a nucleic acid sequence encoding the CLN1 polypeptide of SEQ ID NO: 2.

13. A recombinant adeno-associated virus (rAAV) vector comprising the polynucleotide of any one of claims 1-12.

14. The rAAV vector of claim 13, wherein the rAAV is of the serotype AAV1, AAV2, AAV3, AAV4, AAV5, AAV6, AAV7, AAV8, AAV9, AAVRH10, AAVrh74, AAV11, AAV12, AAV13 or Anc80, AAV7m8 and their derivatives.

15. A recombinant adeno-associated virus (rAAV) particle comprising the polynucleotide of any one of claims 1-12 or the rAAV vector of claim 13 or 14.

16. The rAAV particle of claim 15, wherein the rAAV particle comprising a single-stranded genome.

17. A recombinant adeno-associated virus (rAAV) viral particle encoding a CLN1 polypeptide, comprising an rAAV9 genome comprising in 5′ to 3′ order: a P546 promoter, and a polynucleotide encoding the CLN1 polypeptide.

18. A recombinant adeno-associated virus (rAAV) viral particle encoding a CLN1 polypeptide, comprising an rAAV9 genome comprising in 5′ to 3′ order: a CB promoter, and a polynucleotide encoding the CLN1 polypeptide.

19. A self-complementary recombinant adeno-associated virus (scAAV) comprising the polynucleotide of any one of claims 1-12, the rAAV of claim 13 or 14, or the rAAV particle of any one of claims 15-18.

20. The scAAV of claim 19, wherein the scAAV comprises a single stranded genome.

21. A composition comprising the polynucleotide of any one of claims 1-12, the rAAV vector of claim 13 or 14, the viral particle of any one of claims 15-18 or the scAAV of claim 19 or 20 and a pharmaceutically acceptable excipient, carrier, or diluent.

22. The composition of claim 21, wherein the excipient comprises a non-ionic low osmolar compound.

23. A method of treating CLN1-Batten Disease in an individual comprising administering to the individual a composition comprising the polynucleotide of any one of claims 1-12, the rAAV vector of claim 13 or 14, the viral particle of any one of claims 15-18, the scAAV of claim 19 or 20, or the composition of claim 21 or 22.

24. The method of claim 23, wherein the composition is administered via an intrathecal route, an intracerebroventricular route, an intraperenchymal route, an intravenous route, or a combination thereof.

25. The method of claim 24, wherein the composition is administered intrathecally.

26. The method of claim 24, wherein the composition is administered intracerebroventricularly.

27. The method of claim 24, wherein the composition is administered intravenously.

28. The method of any one of claims 23-27, wherein about 1×10¹³ to about 1×10¹⁵ vg of the scAAV or rAAV viral particle is administered.

29. The method of any one of claims 23-28, further comprising placing the individual in the Trendelenberg position after administering of the scAAV, rAAV viral particle, polynucleotide or the composition.

30. A composition for treating CLN1-Batten Disease in a subject, wherein composition comprises a therapeutically effective amount of the polynucleotide of any one of claims 1-12, the rAAV vector of claim 13 or 14, the viral particle of any one of claims 15-18, the scAAV of claim 19 or 20, or the composition of claim 21 or 22.

31. Use of a therapeutically effective amount of the polynucleotide of any one of claims 1-12, the rAAV vector of claim 13 or 14, the viral particle of any one of claims 15-18, the scAAV of claim 19 or 20, or the composition of claim 21 or 22, for the preparation of a medicament for treating CLN1 Batten Disease.

32. The composition or use of claim 30 or 31, wherein the composition or medicament is formulated for administration via an intrathecal route, an intracerebroventricular route, an intraperenchymal route, an intravenous route, or a combination thereof.

33. The composition or use of claim 32, wherein the composition or medicament is formulated for intrathecal administration.

34. The composition or use of claim 32, wherein the composition or medicament is formulated for intracerebroventricular administration.

35. The composition or use of claim 32, wherein the composition or medicament is formulated for intravenous administration.

36. The composition or use of any one of claims 30-35, wherein the composition or medicament comprises about 1×10¹³ to about 1×10¹⁵ vg of the scAAV or rAAV viral particles.

37. The composition or use of any one of claims 30-36, further comprising placing the individual in the Trendelenberg position after administering of the scAAV, rAAV viral particle, polynucleotide or the composition.
